(19) United States
(12) Patent Application Publication
(10) Pub. No.: US 2024/0122717 A1
(43) Pub. Date: Apr. 18, 2024

(54) ORTHOPEDIC IMPLANT WITH POROUS STRUCTURE AND LATERAL REINFORCEMENT

(71) Applicant: BioMedtrix, LLC, Whippany, NJ (US)

(72) Inventors: Gregory T. Van Der Meulen, Hailey, ID (US); Christopher Preucil, Ketchum, ID (US); Samantha A. Minutillo, Morristown, NJ (US); Christopher G. Sidebotham, Mendham, NJ (US)

(73) Assignee: BioMedtrix, LLC, Whippany, NJ (US)

(21) Appl. No.: 18/486,397

(22) Filed: Oct. 13, 2023

Related U.S. Application Data
(60) Provisional application No. 63/416,297, filed on Oct. 14, 2022.

(51) Int. Cl. A61F 2/36

(52) U.S. Cl. CPC A61F 2/3662; A61F 2002/30909; A61F 2002/3613; A61F 2002/3625

(57) ABSTRACT

A femoral stem implant includes an implant body having a neck and a shaft, the shaft comprising a proximal end portion, a distal end portion, a cranial or anterior aspect, a caudal or posterior aspect, a medial aspect, and a lateral aspect. A porous structure extends circumferentially around the shaft from the cranial or anterior aspect, across the medial aspect, and across the caudal or anterior aspect. At least a portion of the lateral aspect of the shaft comprises a solid reinforcement portion that is at least partially bordered by the porous structure.

### CROSS REFERENCE TO RELATED APPLICATION

The present application claims the benefit of U.S. Provisional Application No. 63/416,297, filed Oct. 14, 2022, which is incorporated herein by reference in its entirety.

### FIELD

The present disclosure pertains to orthopedic implants including porous structures for bone ingrowth and solid reinforcement portions that improve the fatigue resistance of the implants.

### BACKGROUND

Certain orthopedic implants, such as prosthetic joint components used in joint replacement procedures, can be secured in the appropriate bone with cement, or by cementless fixation. In cementless fixation, the implant typically includes a porous structure that engages the native tissue when the implant is received in the prepared bone. For example, in a total hip replacement (hip arthroplasty) procedure, the femoral stem implant can include a porous structure that engages the native tissue of the femoral canal. The preparation is typically undersized relative to the size of the implant to facilitate an interference fit between the implant and the native tissue. The porous structure of the implant promotes stability by limiting movement of the implant relative to the bone to facilitate bone ingrowth. However, porous structures on the implant can be less resistant to cyclic loading, and thus susceptible to fatigue failure. Accordingly, there exists a need for improved orthopedic implants with porous bone ingrowth structures that are resistant to fatigue failure.

### SUMMARY

Certain examples of the disclosure pertain to orthopedic implants such as femoral stem implants including porous structures for bone ingrowth and solid reinforcement portions that extend into the porous structure and are bordered by the porous structure to improve the fatigue resistance of the implants. In a representative example, a femoral stem implant comprises an implant body including a neck and a shaft, the shaft comprising a proximal end portion, a distal end portion, a cranial or anterior aspect, a caudal or posterior aspect, a medial aspect, and a lateral aspect; a porous structure extending circumferentially around the shaft from the cranial or anterior aspect, across the medial aspect, and across the caudal or anterior aspect; and wherein at least a portion of the lateral aspect of the shaft comprises a solid reinforcement portion that is at least partially bordered by the porous structure.

In any or all of the examples described herein, the proximal end portion comprises a solid proximal surface, and the reinforcement portion extends proximally through the porous structure to the proximal surface of the proximal end portion.

In any or all of the examples described herein, the reinforcement portion comprises a solid, machined exterior surface.

In any or all of the examples described herein, the reinforcement portion is bordered on its cranial or anterior longitudinal edge by the porous structure and bordered on its caudal or posterior longitudinal edge by the porous structure.

In any or all of the examples described herein, a proximal edge of the reinforcement portion is bordered by the porous surface.

In any or all of the examples described herein, the femoral stem implant further comprises a bore aligned with an axis of the neck, the bore comprising an opening defined on the lateral aspect of the shaft.

In any or all of the examples described herein, the reinforcement portion surrounds the opening of the bore on the lateral aspect of the shaft.

In any or all of the examples described herein, the reinforcement portion comprises a rib that extends distally from the opening of the bore to a solid portion of the distal end portion of the shaft.

In any or all of the examples described herein, the reinforcement portion comprises a rib that extends proximally from the opening of the bore to a solid proximal surface of the shaft.

In any or all of the examples described herein, the reinforcement portion is an island of solid material around the opening of the bore, and the reinforcement portion is surrounded by the porous structure.

In any or all of the examples described herein, the femoral stem implant comprises a solid substrate on which the porous structure is formed, and the reinforcement portion is a part of the solid substrate.

In another representative example, a femoral stem implant comprises an implant body including a neck and a shaft, the shaft comprising a proximal end portion, a distal end portion, a cranial or anterior aspect, a caudal or posterior aspect, a medial aspect, and a lateral aspect; the implant body comprising a solid substrate and a porous structure formed on the solid substrate, the porous structure extending circumferentially around the shaft from the cranial or anterior aspect, across the medial aspect, and across the caudal or posterior aspect; and a reinforcement portion on the lateral aspect of the shaft, wherein the reinforcement portion is a part of the solid substrate of the implant body and extends proximally through the porous structure such that longitudinal edges of the reinforcement portion are bordered by the porous structure.

In another representative example, an orthopedic implant comprises an implant body extending in a direction of implantation of the orthopedic implant, the implant body comprising a proximal end portion and a distal end portion, the proximal end portion comprising a medial aspect and a lateral aspect; a porous structure extending circumferentially around at least a portion of the proximal end portion of the implant body; and wherein the lateral aspect of the proximal end portion comprises a solid reinforcement portion that is at least partially bounded by the porous structure.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 is a front elevation view of representative examples of orthopedic implants for total hip replacement in an exploded state.

FIG. 2 illustrates the orthopedic implants of FIG. 1 in an assembled state.

FIG. 3 is a micrograph illustrating a three-dimensional porous structure of the acetabular cup of FIG. 2, according to one example.

FIG. 4 is a schematic diagram illustrating the orthopedic implants of FIG. 1 implanted in a canine patient.

FIG. 5 is a schematic diagram illustrating the femoral stem implant of FIG. 1 implanted in a femur, and the forces applied to the implant during activity.

FIG. 6 is a front elevation view of femoral stem implant including a porous structure and a reinforcement portion on a lateral aspect of the shaft.

FIG. 7A is a front elevation view of the femoral stem implant of FIG. 1.

FIG. 7B is a cross-sectional view of the femoral stem implant of FIG. 7A taken along line 7B-7B in FIG. 7A.

FIG. 8A is a front elevation view of the femoral stem implant of FIG. 6.

FIG. 8B is a cross-sectional view of the femoral stem implant of FIG. 8A taken along line 8B-8B in FIG. 8A.

FIGS. 9 and 10 are front elevation and perspective views, respectively, of another example of a femoral stem implant including a porous structure and a reinforcement portion on the lateral aspect.

FIG. 11 is a front elevation view of another example of a femoral stem implant including a bolt extending through a bore aligned with an axis of the neck.

FIGS. 12 and 13 are perspective and side elevation views, respectively, of another example of a femoral stem implant with a reinforcement portion, a bore for receiving a bolt, and with the porous structure removed for purposes of illustration.

FIGS. 14 and 15 illustrate the results of a finite element analysis (FEA) simulation of stresses developed in a femoral stem implant similar to FIG. 11.

FIGS. 16 and 17 illustrate the results of a FEA simulation of stresses developed in the femoral stem implant of FIG. 12.

FIG. 18 is a perspective view of another example of a femoral stem implant including a porous structure and a reinforcement portion extending proximally through the porous structure on the lateral aspect of the shaft.

FIGS. 19-21 are side elevation views of the femoral stem implant of FIG. 6 with various examples of reinforcement portions.

FIG. 22 is a side elevation view of another example of a femoral stem implant including a porous structure and a reinforcement portion on the lateral aspect.

FIGS. 23-26 are side elevation views of the femoral stem implant of FIG. 18 including various examples of reinforcement portions around the opening of the bore in the lateral aspect of the shaft.

FIG. 27 is a side elevation view of the femoral stem implant of FIG. 12 including a wider reinforcement portion around the opening of the bore in the lateral aspect of the shaft.

FIGS. 28 and 29 are perspective and front elevation views, respectively, of the femoral stem implant of FIG. 18 with the porous structure removed for purposes of illustration.

FIG. 30 is a side elevation view of the femoral stem implant of FIG. 28.

FIG. 31 is a cross-sectional front elevation view of the femoral stem implant of FIG. 28.

### DETAILED DESCRIPTION

### Explanation of Terms

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present or problems be solved.

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods.

As used in this disclosure and in the claims, the singular forms “a,” “an,” and “the” include the plural forms unless the context clearly dictates otherwise. Additionally, the term “includes” means “comprises.” Further, the terms “coupled” and “associated” generally mean electrically, electromagnetically, and/or physically (e.g., mechanically or chemically) coupled or linked and does not exclude the presence of intermediate elements between the coupled or associated items absent specific contrary language.

In some examples, values, procedures, or apparatus may be referred to as “lowest,” “best,” “minimum,” or the like. It will be appreciated that such descriptions are intended to indicate that a selection among many alternatives can be made, and such selections need not be better, smaller, or otherwise preferable to other selections.

In the description, certain terms may be used such as “up,” “down,” “upper,” “lower,” “horizontal,” “vertical,” “left,” “right,” and the like. These terms are used, where applicable, to provide some clarity of description when dealing with relative relationships. But, these terms are not intended to imply absolute relationships, positions, and/or orientations. For example, with respect to an object, an “upper” surface can become a “lower” surface simply by turning the object over. Nevertheless, it is still the same object.

Unless otherwise indicated, all numbers expressing angles, dimensions, quantities of components, forces, moments, molecular weights, percentages, times, and so forth, as used in the specification or claims are to be understood as being modified by the term “about.” Accordingly, unless otherwise indicated, implicitly or explicitly, the numerical parameters set forth are approximations that can depend on the desired properties sought and/or limits of detection under test conditions/methods familiar to those of ordinary skill in the art. When directly and explicitly distinguishing embodiments from discussed prior art, the embodiment numbers are not approximates unless the word “about” is recited.

Although there are alternatives for various components, parameters, operating conditions, etc., set forth herein, that does not mean that those alternatives are necessarily equivalent and/or perform equally well. Nor does it mean that the alternatives are listed in a preferred order unless stated otherwise.

As used herein, values modified by the term “substantially” mean±10% of the stated value. The term “substantially parallel” means an angle of ±10° between an object and a reference. The term “substantially perpendicular” means an angle of 80° to 100° between an object and a reference.

### Example 1: Acetabular Cup and Femoral Stem Prostheses for Total Hip Replacement

In a total hip replacement procedure (also known as hip arthroplasty), the articular surfaces of the native hip joint are replaced with prosthetic implants that are secured to the pelvis and the femur to form a ball-and-socket joint. The prosthetic joint assembly typically includes a femoral hip step that is inserted into the canal of the femur, a femoral head typically including a spherical bearing or ball coupled to the femoral hip stem, and an acetabular cup that is inserted into the acetabulum of the pelvis and configured to receive the prosthetic femoral head. In a typical procedure, the femur is separated from the acetabulum, and the native femoral head is removed from the femur. The acetabulum is prepared using a multi-step reaming process to remove tissue and expose healthy bone, typically with a reamer that is smaller than the acetabular cup diameter. The acetabular cup implant is secured in the prepared acetabulum. In certain examples, the cup may be press-fitted into place (e.g., by impact), and/or may be cemented into the socket. The femur is axially reamed (e.g., with a broach) to create an opening into which the femoral stem prosthesis can be inserted. The femoral stem prosthesis can also be press-fitted and/or cemented into place, depending upon the particular indication. The prosthetic femoral head can be attached to the femoral stem prosthesis, and the femur can be maneuvered into place such that the prosthetic femoral head is received in the acetabular cup to form a prosthetic hip joint.

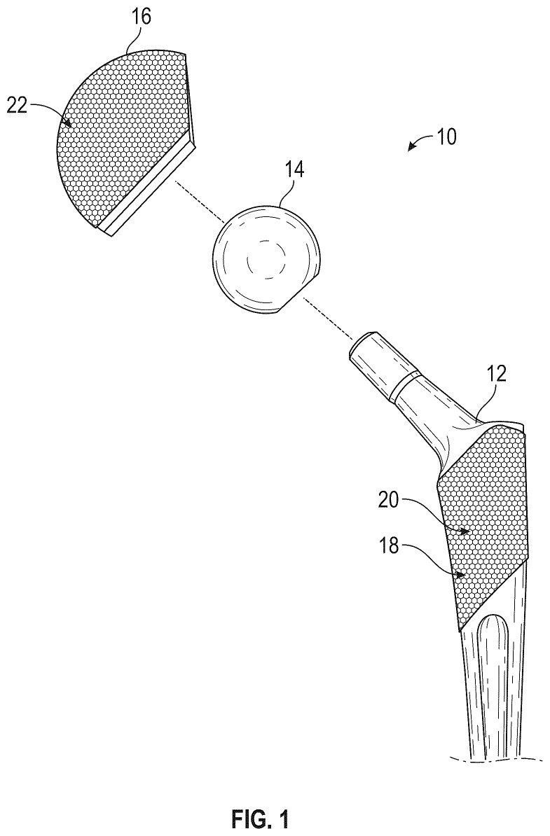

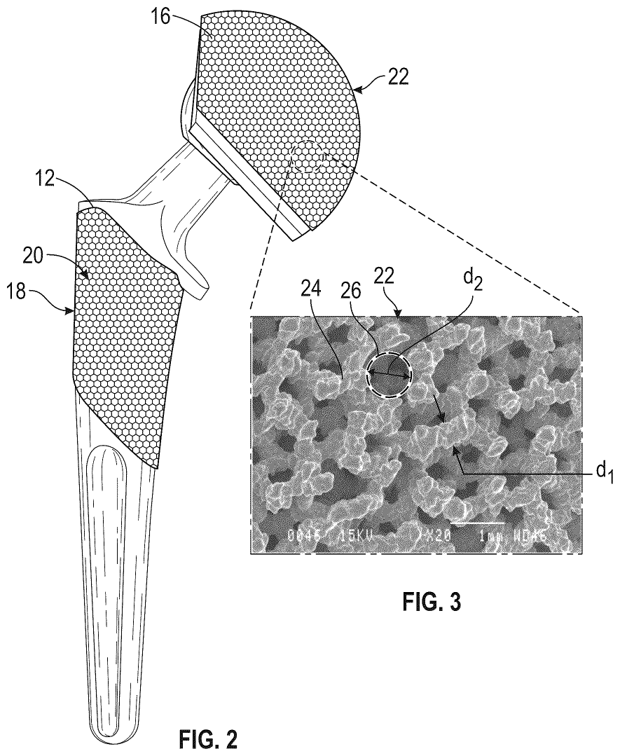

FIG. 1 illustrates an exemplary prosthetic implant assembly 10 for hip arthroplasty including a femoral stem prosthesis 12, a prosthetic femoral head 14, and an acetabular cup 16. FIG. 2 illustrates the components in the assembled state in which the prosthetic femoral head 14 is coupled to the femoral stem prosthesis 12 and received in the acetabular cup 16.

In the embodiment illustrated in FIGS. 1 and 2, the femoral stem prosthesis 12 and the acetabular cup 16 are configured primarily for cementless fixation in the respective native bones. Accordingly, the femoral stem prosthesis 12 includes a region or zone 18 near its proximal end comprising a porous bone-contacting structure 20 configured to engage the tissue of the native femoral canal. The acetabular cup 16 includes a similar porous structure 22 on its exterior surface configured to engage the tissue of the acetabular socket.

In certain examples, the porous structure can comprise a three-dimensional scaffold and/or lattice structure/arrangement comprising a plurality of angled, interconnected strut members. In certain examples, the strut members of the porous structure can be formed on a solid substrate of the implant body and can extend outwardly from the solid substrate surface. The strut members can be arranged to define openings and/or pores between them. For example, FIG. 3 illustrates a representative example of a three-dimensional, porous lattice structure 22 of the acetabular cup 16. The lattice structure can comprise a plurality of angled strut members 24 defining pores, openings, and/or voids 26 between them. In certain examples, the strut members 24 can have a specified diameter d₁, and the pores 26 can have a specified diameter d₂. The diameters d₁ and d₂ can be selected according to any of various factors, such as the type of implant, the implant material, the type of tissue at the interface with the implant, the species and/or size of the patient, etc. In certain examples, the three-dimensional porous structure can be formed by three-dimensional printing and/or additive manufacturing techniques. In certain examples, the diameter d₁ can be 0.1 mm to 0.6 mm, such as 0.2 mm to 0.5 mm, 0.6 mm or less, 0.5 mm or less, etc. In certain embodiments, the diameter d₂ can be 0.1 mm to 2 mm, such as 0.1 mm to 1.5 mm, 0.5 mm to 2 mm, 0.5 mm to 1.5 mm, 0.1 mm to 1 mm, etc.

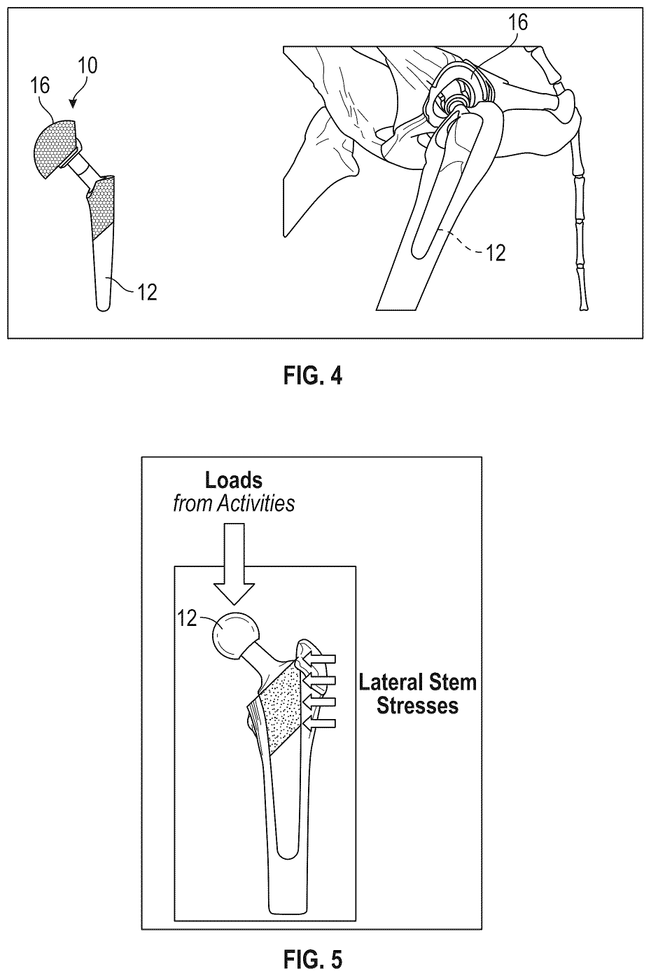

FIG. 4 illustrates a prosthetic hip implant assembly 10 implanted in a canine patient. Although the orthopedic implant examples described herein are configured primarily for veterinary (e.g., non-human) applications, it should be appreciated that the orthopedic implant examples described herein can be configured for both human and veterinary applications.

### Example 2: Femoral Stem Implants with Lateral Reinforcement

After a hip arthroplasty patient recovers from the procedure, the femoral stem is loaded during normal activity (e.g., walking, running, etc.) and stresses can be generated along the lateral side of the stem as shown in FIG. 5. These stresses can create a bending moment which in certain cases can eventually cause the femoral stem to fail from fatigue.

The inventors of the present application have created structural features that advantageously improve the strength of the lateral side (also referred to as the “lateral aspect”) of the stem to better resist stresses and provide a longer service life for the implant.

Manufacturing femoral stems for total hip replacement can be done using one or several of the following manufacturing processes: (1) subtractive machining from bar stock or flat stock material through milling, drilling and turning operations; (2) forging followed by machining; (3) investment casting followed by machining; (4) metal injection molding followed by selectively machining features; (5) powder metal sintering; (6) 3D printing (additive manufacturing from metal powder) including by electron beam melting (EBM) and/or direct laser metal sintering (DLMS).

In all these processes the resultant femoral stem can still be highly stressed in the proximal lateral surface, making them susceptible to fatigue fracture (FIG. 5). Typically, the femoral stem experiences higher stresses during activity than the acetabular cup, making the femoral stem more challenging to produce by additive manufacturing techniques. This can be true especially for titanium alloys, which are notch sensitive. The rough surface finish created by the 3D printing process can create stress risers and/or stress concentrations which can contribute to fatigue failure of the stem. This can be especially problematic when stems are inappropriately sized for the patient (e.g., an undersized stem is implanted in a patient that is heavier or more active than is indicated for the implanted stem).

Increasing the strength of the cross section and improving the surface finish at select locations on the implant as described herein can yield an improved stem design and an increased resistance to fatigue. The features described herein can make femoral stems more versatile, and can allow them to address a wider range of clinical indications for patients. Femoral stems configured as described herein can be used in younger, more active patients, and can be less sensitive to implant sizing issues while retaining the benefits of a three-dimensionally printed porous structure for bone ingrowth and long term stabilization.

As noted above, in certain examples femoral stems can have a proximal porous three-dimensional (3D) structure which allows for bone ingrowth for long-term fixation of the implant to the bone. In certain examples, this porous structure can cover approximately 50% of the stem length in the proximal area and can circumferentially cover that area (e.g., the porous structure extends around the entire circumference of the proximal portion of the shaft). The lateral surface of the stem typically experiences the highest stresses and, given the roughness of the surfaces created through 3D printing and the porous structure, the lateral surface can be susceptible to fatigue crack initiation. One approach to reduce these stresses is to create a solid surface on the lateral aspect of the stem. This can be done as a solid structure joining the distal portion of the solid stem and continuing proximally to the collar area. The more proximal portion of the stem can have a larger cross section and therefore a larger moment of inertia to resist the stresses imposed during activities.

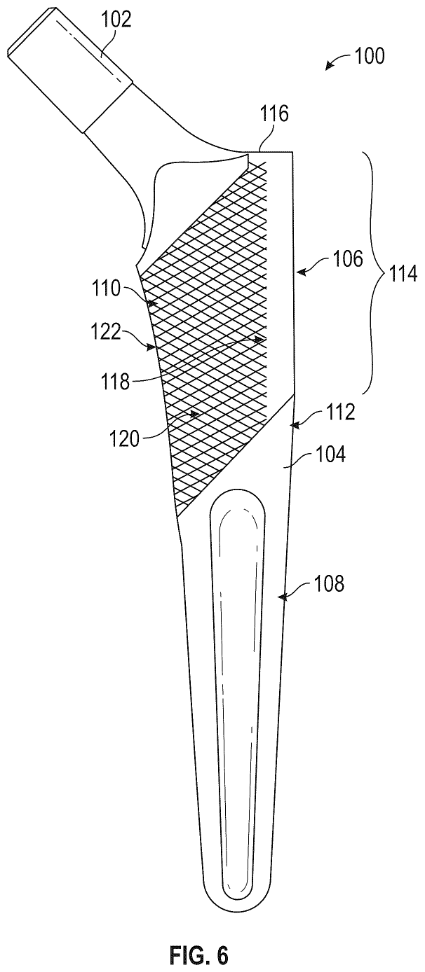

For example, FIG. 6 illustrates a femoral stem implant 100 including a neck 102, a shaft 104 having a proximal end portion 106 and a distal end portion 108, and a porous structure 110 on the proximal end portion 106 of the shaft. The lateral aspect 112 of the proximal end portion 106 has a reinforcement portion 114 (also referred to as a “reinforcement”) including a solid surface that extends from the solid distal end portion 108 of the shaft 104 to the collar area 116. The porous structure 110 extends from a first longitudinal edge (e.g., a cranial or anterior longitudinal edge) 118 of the reinforcement portion across the cranial aspect/surface 120 of the implant body, across and/or around the medial aspect/surface 122 of the implant body, and across the caudal or posterior aspect/surface (not visible) of the implant body to a second longitudinal edge of the reinforcement (e.g., a caudal or posterior longitudinal edge).

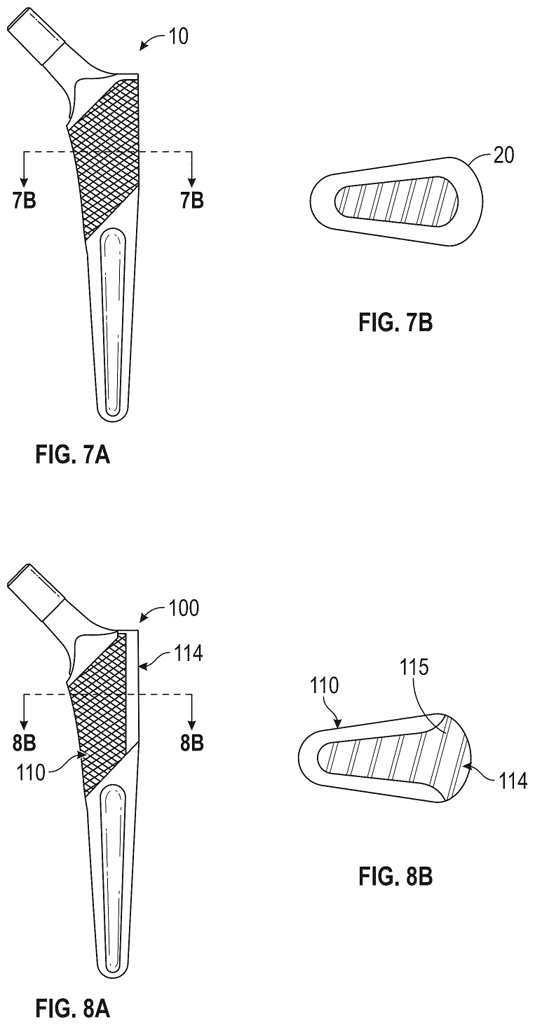

FIGS. 7A-7B illustrate a cross-section taken along line 7B-7B (FIG. 7A) through the neck of a femoral stem 10 with a porous structure 20 that is continuous around the circumference of the proximal portion of the shaft and lacks a reinforcement. FIGS. 8A and 8B illustrate a cross-section through the proximal portion of a shaft of a femoral stem 100 including one example of a reinforcement portion 114 as described herein. The reinforcement portion 114 can improve the fatigue resistance of the stem because there is an increased area of the solid substrate 115. The increased area of solid substrate can increase the moment of inertia of the stem, making the stem more resistant to cyclic fatigue failure. The increased solid substrate area of the reinforcement portion can also reduce the lateral stress developed in the stem for a given force/load. The exterior surface of the reinforcement portion 114 can also have a machined surface finish (e.g., the surface can be processed to remove the rough 3D printed surface finish). This can improve fatigue resistance of the stem at the location of the reinforcement portion 114 by significantly reducing or eliminating the notch sensitivity associated with the rough surface of the porous structure 110. Stated differently, the machined exterior surface of the reinforcement portion 114 can reduce the incidence of crack initiation and/or crack propagation under normal loading conditions. In contrast, the three-dimensionally printed implant 10 in FIG. 7A has a solid substrate with a rough surface around a larger proportion of the lateral aspect of the shaft, which can be more susceptible to fatigue crack initiation. With a 3D printed porous structure, the surface finish of the underlying substrate typically cannot be improved.

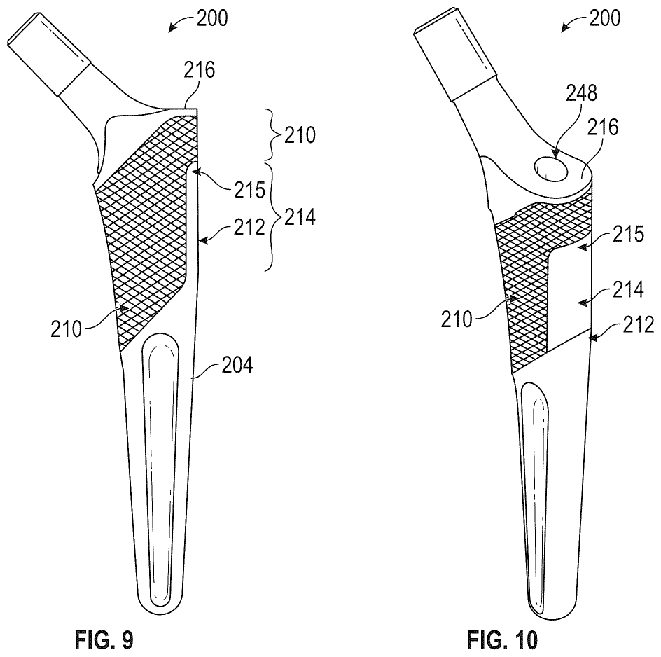

The reinforcement portion can be shaped and sized in a variety of ways. For example, the reinforcement portion can be configured as an elongated portion of solid material extending in a proximal-distal direction along the lateral aspect of the implant. In certain examples, the lateral aspect of the implant can be solid, and the exterior surface of the solid lateral aspect can be machined and/or polished to a specified surface finish. For example, the surface can be left “as printed” (e.g., the surface finish produced by the additive manufacturing system without further processing), bead blasted, machined, machined and polished, etc. In certain examples, the reinforcement portion can be created by extending the solid substrate of the implant to the exterior surface of the implant. In certain examples, the reinforcement portion can extend from the solid distal portion of the shaft to the collar of the implant at the proximal end of the shaft as in FIGS. 6 and 8A-8B. In certain other examples such as the femoral stem implant 200 in FIGS. 9 and 10, the reinforcement portion 214 can be shorter such that there is a gap between the proximal end 215 of the reinforcement portion 214 and the collar 216. The porous structure 210 can extend continuously around the full perimeter of the shaft 204 in the region between the end of the reinforcement portion 214 and the collar 216. Stated differently, a portion of the lateral aspect 212 of the proximal shaft 204 can be solid, and a portion of the lateral aspect 212 that is proximal of the solid region 214 can have a 3D porous structure. In some examples the stem 200 can include a recess (also referred to as a divot or indentation) 248 defined in the proximal surface of the collar portion 216 of the shaft 204. The recess 248 can be sized and shaped to receive the distal end of an impactor, which can be used to drive the femoral stem into the prepared femoral canal during implantation.

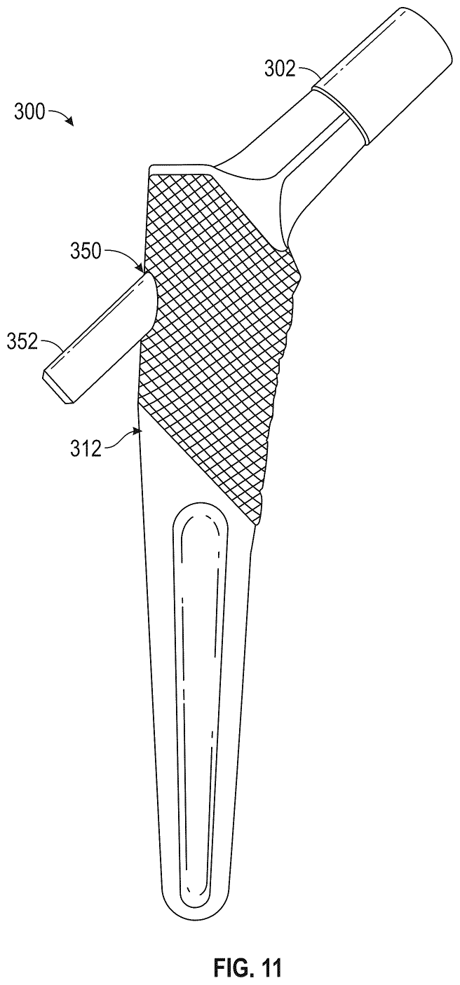

In another example shown in FIG. 11, a femoral stem 300 can define an opening 350 aligned with the axis of the neck 302 through which a bolt 352 (also referred to as a “lateral bolt”) can be inserted in the lateral side 312 of the stem for improved torsional resistance. Adding a lateral solid substrate (e.g., reinforcement portion) around the hole 350 and along the lateral side 312 of the stem can improve the fatigue resistance of the stem and at least partially compensate for weakening of the stem due to the through hole 350.

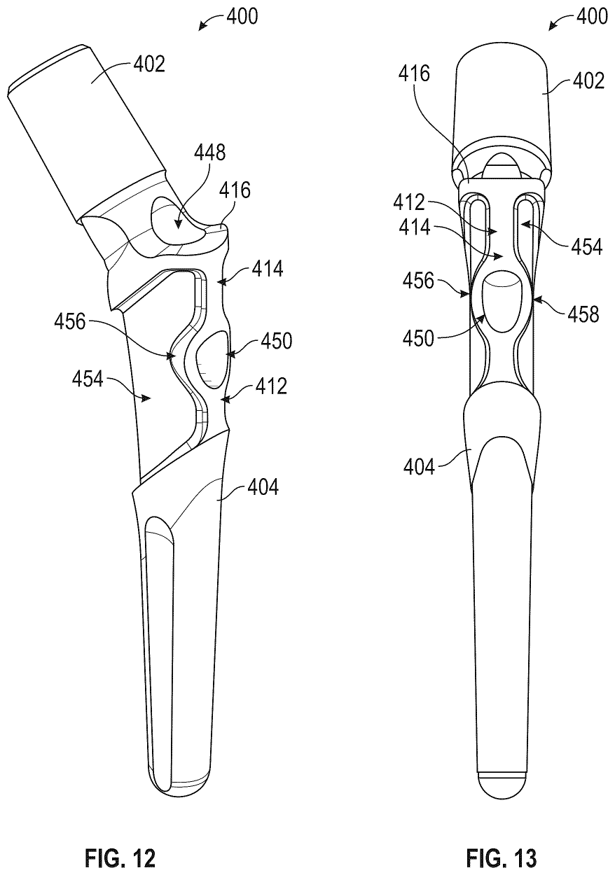

FIGS. 12 and 13 illustrate the solid substrate of a femoral stem implant body 400 including a passage (also referred to as a bore) 450 extending through the lateral aspect 412 of the shaft 404 and aligned with the axis of the neck 402 similar to the implant of FIG. 11. In FIGS. 12 and 13 the implant 400 is shown without the porous structure. Stated differently, only the solid (e.g., non-porous) substrate portions of the implant 400 are shown in FIGS. 12 and 13. The reinforcement portion 414 can be configured as a rib that extends in the proximal-distal direction and surrounds the opening of the bolt hole on the lateral surface 412. In the complete implant the porous structure is built up from the solid surface in the depression (also referred to as a recessed portion) 454 that extends from the first longitudinal edge 456 (e.g., the cranial or anterior edge) of the reinforcement portion 414 around the shaft to the second longitudinal edge 458 (e.g., the caudal or posterior edge). The reinforcement portion 414 can extend outwardly from the recess 454 of the solid substrate. The implant can also include a round recess 448 formed in the proximal surface of the collar portion 416 of the shaft 404.

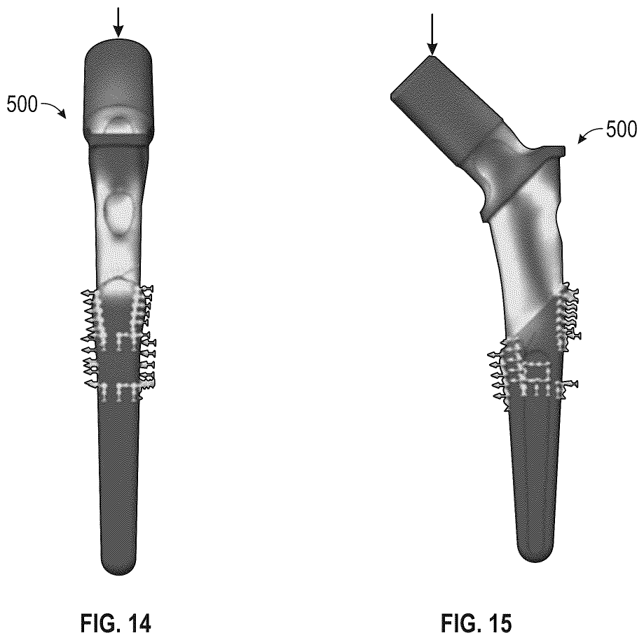

Finite element analysis (FEA) of the lateral bolt stem design including a reinforcement portion as shown in FIGS. 12-13 illustrates the potential improvement from including a lateral reinforcement portion. By creating a solid substrate reinforcement around the lateral hole instead of the hole passing through a porous 3D structure, there is a substantial improvement in surface finish. This improvement from an as-printed surface roughness to a post-processed surface can result in a 300% to 400% improvement in fatigue life. The output of an FEA simulation of an existing femoral stem 500 with a bolt hole opening surrounded by a 3D porous structure is shown in FIGS. 14 and 15. The femoral stem 500 had a maximum yield force/mass of 184 lbs (83.5 kg).

When a porous structure is used in the lateral area of the stem, it does not provide the opportunity to improve the surface finish of the substrate beneath the porous structure nor address the notch sensitivity created by the attachment of the struts within the 3D porous ingrowth structure to the substrate. The femoral stem examples described herein provide an alternative in which a portion of the porous structure (or all of the porous structure along the lateral side of the stem) is replaced with a solid substrate having a specified surface finish, resulting in an improved fatigue life of the stem. This reduction in porous structure can still provide adequate bone ingrowth area for long term stabilization of the implant.

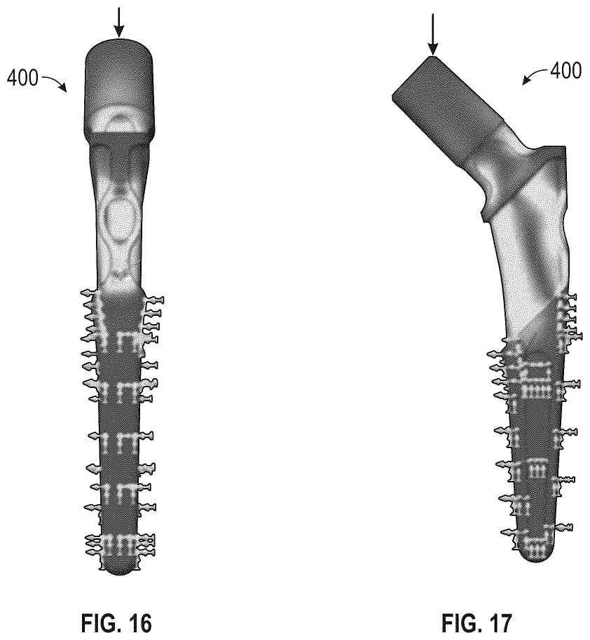

FIGS. 16 and 17 illustrate the output of an FEA simulation of an example of a femoral stem similar to FIGS. 12-13 including a lateral reinforcement rib and an “island” of solid substrate material around the bolt hole. As noted above, this stem example provides an increased solid surface area and increased moment of inertia along the lateral/side aspect of the stem shaft. As shown in FIGS. 16 and 17, incorporating the reinforcement portion reduces the lateral stem stress by 17% as compared to the unreinforced configuration in FIGS. 14 and 15. As shown in the FEA results, the example in FIGS. 16 and 17 achieves a 17% increase in the maximum yield force/mass of the implant to 215 lbs (97.5 kg), indicating improved stem strength and ability to resist fatigue fracture of over 17% based on geometry, which can be in addition to the fatigue improvement attributable to the surface finish.

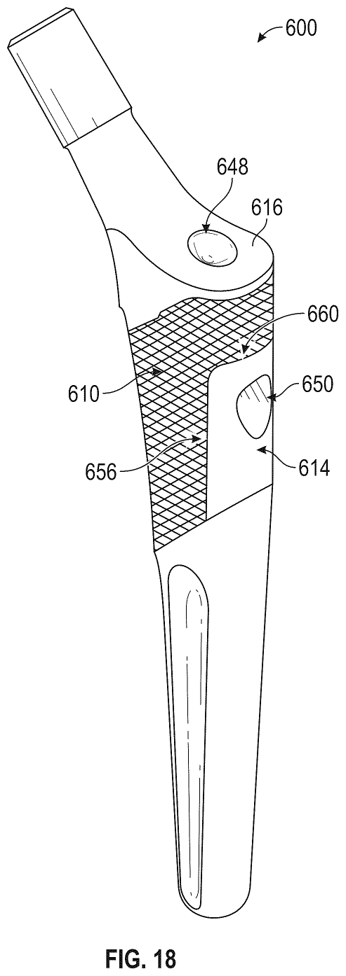

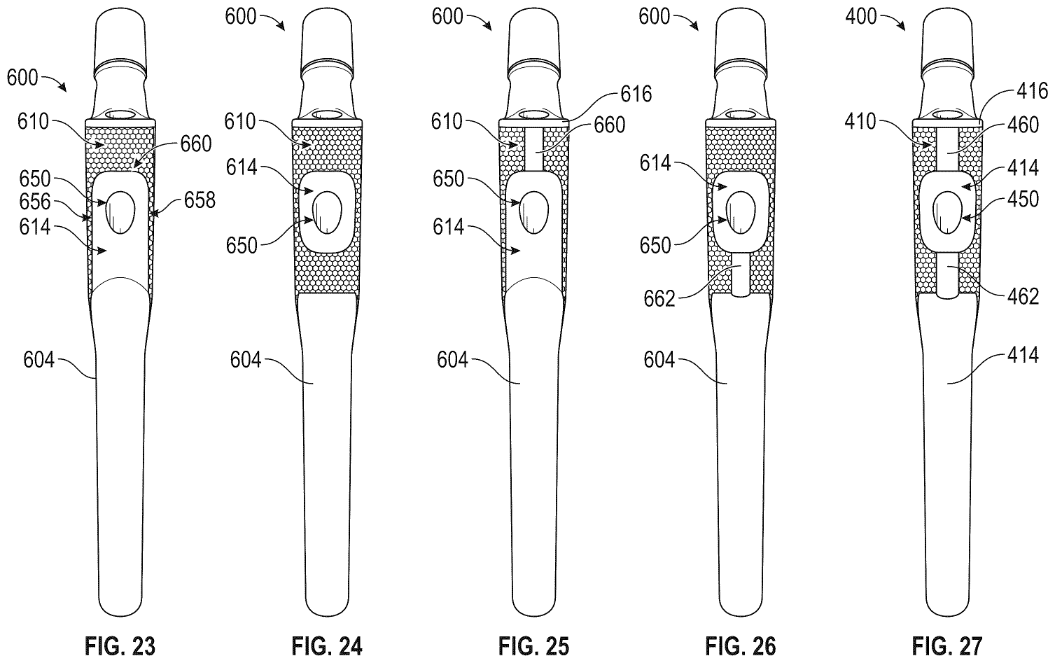

FIG. 18 illustrates another example of a femoral stem implant 600 similar to FIGS. 9 and 10 in which the reinforcement portion 614 is bounded on its cranial (first and/or anterior) edge 656, caudal (second and/or posterior) edge 658 (FIG. 23), and ventral (proximal) edge 660 by the porous structure 610, and which includes a bolt hole 650 through the reinforcement portion 614. Stated differently, the reinforcement portion 614 is bordered by the porous structure 610 along the cranial/anterior edge 656, the caudal/posterior edge 658, and the proximal edge 660 of the reinforcement portion 614. The increased surface area of the solid lateral substrate (e.g., the reinforcement portion surface area) relative to the example of FIGS. 12-13 can provide a corresponding increase in fatigue resistance. The implant 600 can also include a round recess 648 in the collar portion 616 of the shaft 604.

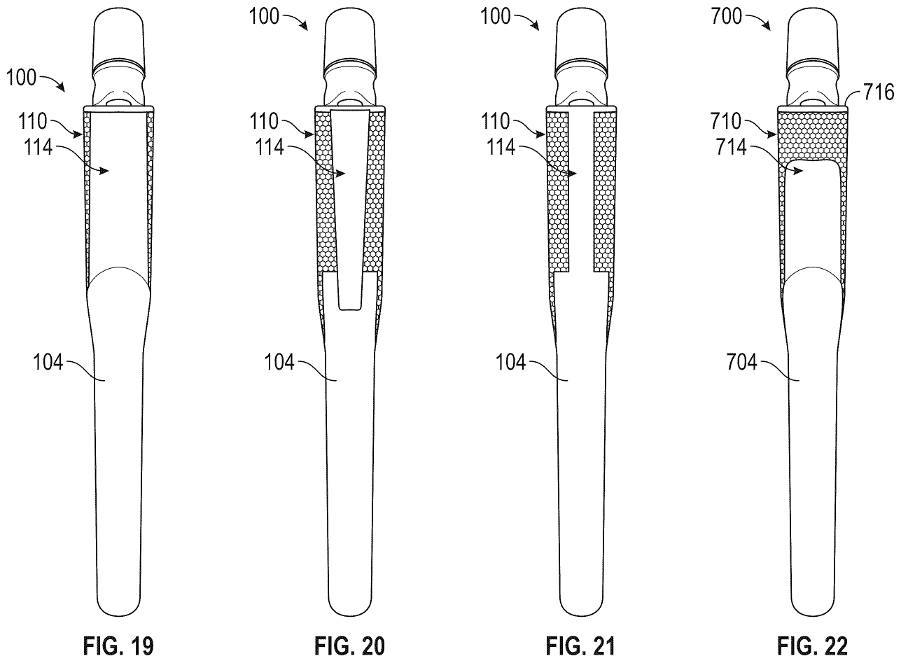

FIGS. 19-22 illustrate various examples of femoral stem implants with circumferentially extending porous structures on the proximal portion of the shaft, and which include reinforcement portions on the lateral aspect wherein the reinforcement portion is a region of solid material having a machined/polished exterior surface extending in the proximal-distal direction and bounded along at least two edges by the porous structure. The reinforcement portion can have various shapes, lengths, widths, etc. The machined/polished exterior surface of the reinforcement portion can be continuous with the machined/polished exterior surface of the distal end portion of the shaft.

FIG. 19 illustrates a side elevation view of the femoral stem implant 100 of FIG. 6. FIG. 20 illustrates another example of the femoral stem implant 100 in which the longitudinal edges of the solid reinforcement portion 114 are angled such that the reinforcement portion is tapered in the distal direction. Stated differently, the solid reinforcement portion 114 in FIG. 20 is wider at its proximal end than at its distal end. FIG. 21 illustrates another example of the femoral stem implant 100 in which the reinforcement portion 114 is narrower than in FIGS. 6 and 19 and the porous structure 110 extends around a greater proportion of the perimeter of the proximal portion of the shaft 104.

FIG. 22 is a side elevational view of a femoral stem implant 700 including a reinforcement portion 714 that extends upwardly from the solid distal portion of the shaft 704 and is surrounded on three sides by a porous structure 710 similar to the implants of FIGS. 10 and 18 but without a bolt hole. A continuous band of the porous structure 710 encircles the proximal portion of the shaft 704 adjacent the collar 716.

FIGS. 23-27 illustrate various examples of femoral stem implants with circumferentially extending porous structures on the proximal portion of the shaft, and which include a reinforcement portion on the lateral aspect configured as a region of solid material having a machined/polished exterior surface. The stem implants in FIGS. 23-27 can comprise bolt holes with openings located in the reinforcement portion on the lateral aspect. In certain examples the reinforcement portion can extend in the proximal-distal direction. In certain examples the reinforcement portion can be bounded along at least two edges by the porous structure. In certain examples, the reinforcement portion can be an “island” surrounded by the porous structure. In certain examples the island reinforcement portion can include a reduced width portion extending distally to the smooth solid distal end portion of the shaft, and/or a reduced width portion extending proximally to the collar at the proximal end of the shaft.

For example, FIG. 23 illustrates a side elevational view of the femoral stem 600 of FIG. 18. FIG. 24 illustrates another example of the femoral stem 600 in which the reinforcement portion 614 is an island of solid material surrounded by the porous structure 610. The solid material of the reinforcement portion 614 surrounds the lateral opening of the bore 650. FIG. 25 illustrates another example of the femoral stem 600 in which the reinforcement portion 614 includes a relatively narrow portion or rib 660 of material that extends proximally from the main reinforcement portion through the porous structure 610 to the collar portion 616. FIG. 26 illustrates another configuration of the example shown in FIG. 24 in which a rib 662 of solid material extends from the island reinforcement 614 distally through the porous structure 610 to the solid distal portion of the shaft 604.

FIG. 27 illustrates another example of the femoral stem 400 in which the solid reinforcement material around the lateral opening of the bore 450 is wider than the configuration shown in FIGS. 12 and 13. A rib 460 extends proximally through the porous structure 410 to the collar 416 and a rib 462 extend distally through the porous structure 410 to the solid distal portion of the shaft 404.

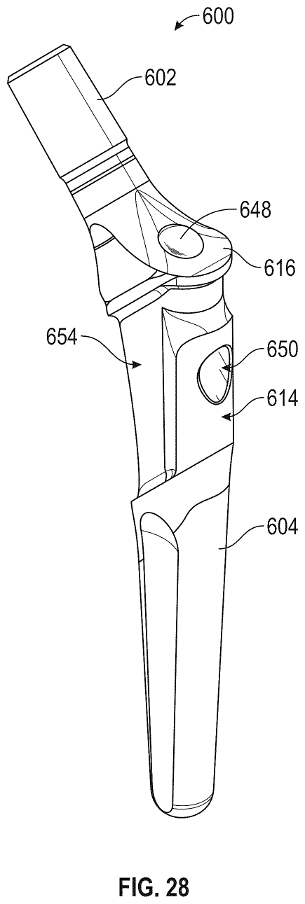

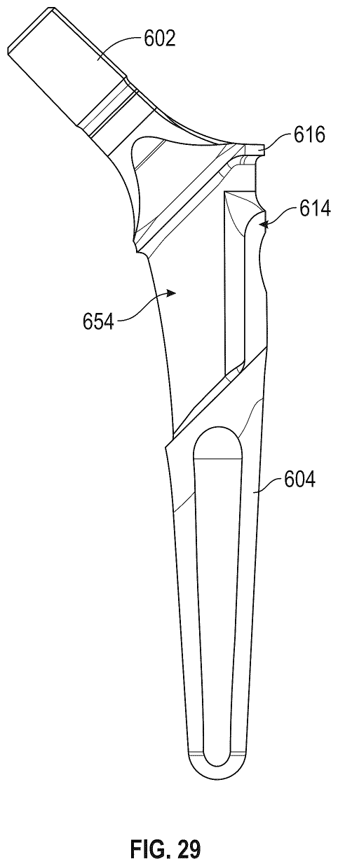

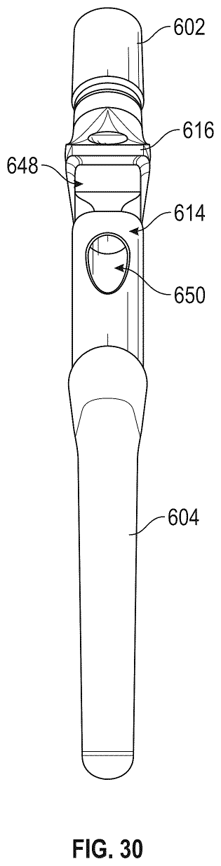

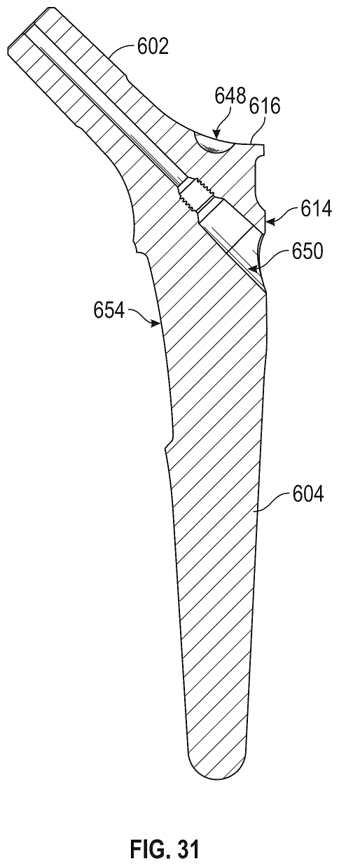

FIGS. 28-31 are additional views illustrating the femoral stem of FIG. 23 without the porous structure for purposes of illustration. Stated differently, FIGS. 28-31 show the solid substrate of the implant 600 without the porous structure 610. The porous structure can be formed on the solid surface of the recess 654 defined in the proximal portion of the shaft 604 and shown extending around the perimeter of the implant and around the reinforcement portion 614. As shown in FIG. 31, the bore 650 can extend along the axis of the neck 602 and can be configured to receive a lateral bolt similar to the lateral bolt 352.

In certain embodiments the porous structure can be configured such that it exhibits a coefficient of friction with bone tissue that varies along the surface of the porous structure. Such variation can be continuous along the surface of the porous structure in a particular direction (e.g., in the direction of implantation), or in discrete regions or zones exhibiting different coefficients of friction with bone tissue. In certain examples the porous structure can gradually develop or “fade in” from a solid substrate in a selected direction, such as by employing struts with diameters that decrease in the selected direction until the porous structure is fully developed. Further details of such porous structures can be found in U.S. patent application Ser. No. 18/485,626, which is incorporated herein by reference in its entirety.

The femoral stem prosthesis examples described herein can be formed of any of various biocompatible metal materials. For example, in certain examples the prostheses can be formed of titanium alloys, such as ASTM F-136 (Ti-6Al-4V ELI Titanium Alloy). In other examples the femoral stem prostheses can be formed using other biocompatible metals such as cobalt chromium alloys, stainless steel alloys, and/or various composite materials or polymers. As noted above, the femoral stem implants described herein can be fabricated using additive manufacturing techniques such as direct laser metal sintering and/or electron beam melting to form the solid substrate, the porous structure, and the reinforcement portion. As noted above, the reinforcement portion examples described herein can significantly increase the fatigue resistance of such additively manufactured femoral stem implants.

When the implant bodies are formed by additive manufacturing techniques, the solid substrate can be a solid mass of fused raw material (e.g., metal powder). In other examples, the solid substrates can be formed by machining a workpiece of stock material (e.g., metal bar stock) and forming the porous structure on the solid substrate using other techniques.

In view of the many possible embodiments to which the principles of the disclosed technology may be applied, it should be recognized that the illustrated embodiments are only examples and should not be taken as limiting the scope of the disclosure. Rather, the scope of the disclosure is at least as broad as the following claims and equivalents of the recited features. We therefore claim as all that comes within the scope and spirit of these claims.

1. A femoral stem implant, comprising:
an implant body including a neck and a shaft, the shaft comprising a proximal end portion, a distal end portion, a cranial or anterior aspect, a caudal or posterior aspect, a medial aspect, and a lateral aspect;
a porous structure extending circumferentially around the shaft from the cranial or anterior aspect, across the medial aspect, and across the caudal or anterior aspect; and
wherein at least a portion of the lateral aspect of the shaft comprises a solid reinforcement portion that is at least partially bordered by the porous structure.

2. The femoral stem implant of claim 1, wherein the proximal end portion comprises a solid proximal surface, and the reinforcement portion extends proximally through the porous structure to the proximal surface of the proximal end portion.

3. The femoral stem implant of claim 1, wherein the reinforcement portion comprises a solid, machined exterior surface.

4. The femoral stem implant of claim 1, wherein the reinforcement portion is bordered on its cranial or anterior longitudinal edge by the porous structure and bordered on its caudal or posterior longitudinal edge by the porous structure.

5. The femoral stem implant of claim 1, wherein a proximal edge of the reinforcement portion is bordered by the porous surface.

6. The femoral stem implant of claim 1, further comprising a bore aligned with an axis of the neck, the bore comprising an opening defined on the lateral aspect of the shaft.

7. The femoral stem implant of claim 6, wherein the reinforcement portion surrounds the opening of the bore on the lateral aspect of the shaft.

8. The femoral stem implant of claim 7, wherein the reinforcement portion comprises a rib that extends distally from the opening of the bore to a solid portion of the distal end portion of the shaft.

9. The femoral stem implant of claim 7, wherein the reinforcement portion comprises a rib that extends proximally from the opening of the bore to a solid proximal surface of the shaft.

10. The femoral stem implant of claim 7, wherein the reinforcement portion is an island of solid material around the opening of the bore, and the reinforcement portion is surrounded by the porous structure.

11. The femoral stem implant of claim 1, wherein the femoral stem implant comprises a solid substrate on which the porous structure is formed, and the reinforcement portion is a part of the solid substrate and extends outwardly from the solid substrate and proximally through the porous structure.

12. A femoral stem implant, comprising:
an implant body including a neck and a shaft, the shaft comprising a proximal end portion, a distal end portion, a cranial or anterior aspect, a caudal or posterior aspect, a medial aspect, and a lateral aspect;
the implant body comprising a solid substrate and a porous structure formed on the solid substrate, the porous structure extending circumferentially around the shaft from the cranial or anterior aspect, across the medial aspect, and across the caudal or posterior aspect; and
a reinforcement portion on the lateral aspect of the shaft, wherein the reinforcement portion is a part of the solid substrate of the implant body and extends outwardly from the solid substrate and proximally through the porous structure such that longitudinal edges of the reinforcement portion are bordered by the porous structure.

13. The femoral stem implant of claim 12, wherein the proximal end portion comprises a solid proximal surface, and the reinforcement portion extends proximally through the porous structure to the proximal surface of the proximal end portion.

14. The femoral stem implant of claim 12, wherein the reinforcement portion comprises a solid, machined exterior surface.

15. The femoral stem implant of claim 12, wherein the reinforcement portion is bordered on its cranial or anterior longitudinal edge by the porous structure and bordered on its caudal or posterior longitudinal edge by the porous structure.

16. The femoral stem implant of claim 12, wherein a proximal edge of the reinforcement portion is bordered by the porous surface.

17. The femoral stem implant of claim 12, further comprising a bore aligned with an axis of the neck, the bore comprising an opening defined on the lateral aspect of the shaft, and wherein the reinforcement portion surrounds the opening of the bore on the lateral aspect of the shaft.

18. The femoral stem implant of claim 17, wherein the reinforcement portion comprises a rib that extends distally from the opening of the bore to a solid portion of the distal end portion of the shaft.

19. The femoral stem implant of claim 17, wherein the reinforcement portion comprises a rib that extends proximally from the opening of the bore to a solid proximal surface of the shaft.

20. The femoral stem implant of claim 17, wherein the reinforcement portion is an island of solid material around the opening of the bore, and the reinforcement portion is surrounded by the porous structure.
